(19) United States
(12) Patent Application Publication
(10) Pub. No.: US 2026/0216409 A1
(43) Pub. Date: Jul. 30, 2026

(54) POLYMER COMPOSITE FOR URETERAL INSERTION, METHOD FOR PREPARING SAME, AND URETERAL STENT USING SAME

(71) Applicants: SAMSUNG LIFE PUBLIC WELFARE FOUNDATION, Seoul (KR); RESEARCH & BUSINESS FOUNDATION SUNGKYUNKWAN UNIVERSITY, Suwon-si (KR)

(72) Inventors: Deok Hyun HAN, Seoul (KR); Jae Hoon CHUNG, Seoul (KR); Dong Geun JUNG, Seoul (KR); Yoon Soo PARK, Suwon-si (KR); Hyun A LIM, Suwon-si (KR); Nam Wuk BAEK, Suwon-si (KR); Yong Ki CHO, Ansan-si (KR)

(21) Appl. No.: 19/127,944

(22) PCT Filed: Jun. 2, 2023
(86) PCT No.: PCT/KR2023/007584

§ 371 Date: Apr. 6, 2026

(30) Foreign Application Priority Data
Nov. 7, 2022 (KR) 10-2022-0147171

(51) Int. Cl. A61L 31/06; A61L 31/10

(52) U.S. Cl. CPC A61L 31/06; A61L 31/10; A61L 2420/02

(57) ABSTRACT

The present invention relates to a polymer composite for ureteral insertion, a method for preparing the same, and a ureteral stent using the same, and, in particular, to: a polymer composite for ureteral insertion in which the surface of a polymer substrate used in a ureteral stent is treated with plasma to improve the hydrophobicity of the surface, and thus, formation of stones on the surface of the stent can be prevented; a method for preparing the same; and a ureteral stent using the same.

### TECHNICAL FIELD

The present invention relates to a polymer composite for ureteral insertion, a method for preparing the same, and a ureteral stent using the same, and, in particular, to: a polymer composite for ureteral insertion in which the surface of a polymer substrate used in a ureteral stent is treated with plasma to improve the hydrophobicity of the surface, and thus, formation of stones on the surface of the stent can be prevented; a method for preparing the same; and a ureteral stent using the same.

### BACKGROUND ART

Ureteral stents are used as a main means for treating urolithiasis and disorders of genitourinary tract, and for treating fistulas or ureteral injuries caused by trauma. It is important for such ureteral stents to prevent stent-related complications such as stone formation and infection, to suppress encrustation and biofilm (EBF) formation by maintaining patency in genitourinary tract for a long time, and to maintain biocompatibility. However, ureteral stents have problems in that calcium salts, microorganisms and proteins adhere thereto when deposited in urine for a long period of time, producing disorders or stones, and therefore, replacement through surgery is required every 2 to 3 months.

Furthermore, due to the EBF, infection and the like, it has been sometimes necessary for many patients to replace ureteral stents earlier than the replacement cycle. A number of studies have been conducted to resolve this problem, however, most of the studies have been conducted using a method of coating the outside of a stent with silver, hydrogel and antimicrobial peptide.

Accordingly, studies on techniques for suppressing encrustation and biofilm formation by treating the inside of a stent have been required.

### DISCLOSURE

### Technical Problem

An object of the present invention is to provide a polymer composite for ureteral insertion capable of preventing formation of stones, which are foreign substances, occurring over time on the surface of a stent inserted into a ureter as the stent is immersed in urine, a method for preparing the same, and a ureteral stent using the same.

However, objects of the present invention are not limited to the above-mentioned objects, and other objects not mentioned will be clearly appreciated by those skilled in the art from the following description.

### Technical Solution

One aspect of the present invention provides a polymer composite for ureteral insertion, the polymer composite including: a polymer substrate; and a coating layer provided on at least one surface of the polymer substrate, wherein the coating layer has a C atom in an amount of 78 at. % or greater and 82 at. % or less and an O atom in an amount of 13 at. % or greater and 17 at. % or less, which are detected by an analysis using an XPS analysis method.

In one embodiment of the present invention, the coating layer may have an N atom in an amount of 0.5 at. % or greater and 2.5 at. % or less and a Si atom in an amount of 2.5 at. or greater and 4.5 at. % or less, which are detected by an analysis using an XPS analysis method.

In one embodiment of the present invention, the coating layer may have a maximum peak of C 1s in a range of 280 eV or greater and 290 eV or less, a maximum peak of O 1s in a range of 530 eV or greater and 540 eV or less, a maximum peak of N 1s in a range of 390 eV or greater and 410 eV or less and a maximum peak of Si 2p in a range of 100 eV or greater and 100 eV or less, which are detected by an analysis using an XPS analysis method.

In one embodiment of the present invention, the coating layer may have a full width at half maximum of the C 1s peak in a range of 1.2 eV or greater and 1.5 eV or less, a full width at half maximum of the O 1s peak in a range of 1.45 eV or greater and 1.6 eV or less and a full width at half maximum of the N 1s peak in a range of 1.5 eV or greater and 1.8 eV or less, which are detected by an analysis using an XPS analysis method.

In one embodiment of the present invention, the coating layer may have a full width at half maximum of the Si 2p peak detected by an analysis using an XPS analysis method in a range of 1.2 eV or greater and 1.4 eV or less.

In one embodiment of the present invention, the coating layer may have an average thickness of 0.1 μm or greater and 2.0 μm or less.

In one embodiment of the present invention, a material of the polymer substrate may be a polyurethane-based resin.

Another aspect of the present invention provides a ureteral stent formed with the polymer composite for ureteral insertion, wherein the polymer substrate has a tube shape.

In one embodiment of the present invention, the coating layer may be provided inside the tube.

In one embodiment of the present invention, the ureteral stent may have an outer diameter of 1.6 mm or greater and 4.0 mm or less.

In one embodiment of the present invention, the ureteral stent may have an inner diameter of 0.2 mm or greater and 1.7 mm or less.

In one embodiment of the present invention, the coating layer may have 1 or more and 5 or less layers.

Another aspect of the present invention provides a method for preparing the polymer composite for ureteral insertion, the method including: providing a polymer substrate; and providing a coating layer on at least one surface of the polymer substrate by performing plasma deposition for 3 minutes or longer and 5 minutes or shorter under a mixed gas atmosphere including C₂H₂.

In one embodiment of the present invention, the mixed gas may further include one selected from the group consisting of an inert gas, O₂ and combinations thereof.

In one embodiment of the present invention, the inert gas may be one selected from the group consisting of He, Ne, Ar, Kr, Xe, Rn and combinations thereof.

In one embodiment of the present invention, a voltage applied for plasma generation in the plasma deposition may be 1,000 V or greater and 5,000 V or less.

In one embodiment of the present invention, a current applied for plasma generation in the plasma deposition may be 0.1 A or greater and 10.0 A or less.

In one embodiment of the present invention, total energy of plasma generated in the plasma deposition may be 200 J or greater and 30,000 J or less.

In one embodiment of the present invention, a pressure of the mixed gas atmosphere in the plasma deposition may be 0.1 torr or greater and 10.0 torr or less at 20° C.

In one embodiment of the present invention, a flow rate of the mixed gas in the plasma deposition may be 5 sccm or greater and 100 sccm or less.

In one embodiment of the present invention, average molar energy applied to the mixed gas in the plasma deposition may be 1.0×10⁸ J/mol or greater and 1.0×10¹¹ J/mol or less.

### Advantageous Effects

A polymer composite for ureteral insertion according to one embodiment of the present invention is capable of preventing stone substances, which are foreign substances, from being deposited on the surface by improving hydrophobicity of the surface of a polymer substrate.

A ureteral stent according to one embodiment of the present invention is capable of improving durability by preventing stone formation due to a coating layer on the inner surface, and can be used for a long time without replacement.

A method for preparing the polymer composite for ureteral insertion according to one embodiment of the present invention is capable of preventing stone substances, which are foreign substances, from being deposited on the surface by readily forming a coating layer and adjusting a thickness of the coating layer.

Effects of the present invention are not limited to the above-described effects, and effects not mentioned herein will be clearly appreciated by those skilled in the art from the present specification and accompanying drawings.

### BRIEF DESCRIPTION OF DRAWINGS

FIG. 1 is a schematic diagram of a polymer composite for ureteral insertion according to one embodiment of the present invention.

FIG. 2 is a schematic diagram of a ureteral stent according to one embodiment of the present invention.

FIG. 3 is a flow chart of a method for preparing the polymer composite for ureteral insertion according to one embodiment of the present invention.

FIG. 4 is a graph showing an XPS (X-ray photoelectron spectroscopy) analysis result of a polymer substrate layer.

FIG. 5 is a graph showing an XPS (X-ray photoelectron spectroscopy) analysis result of Reference Example 1.

FIG. 6 is a graph showing an XPS (X-ray photoelectron spectroscopy) analysis result of Reference Example 2.

FIG. 7 is a graph showing an XPS (X-ray photoelectron spectroscopy) analysis result of Example 1 according to one embodiment of the present invention.

FIG. 8 is a graph showing an XPS (X-ray photoelectron spectroscopy) analysis result of Example 2 according to one embodiment of the present invention.

FIG. 9 shows photographs of a cross-section taken at a magnification of 150 times after immersing Example 1 according to an embodiment of the present invention in urine for 1 day, 3 days, 5 days, 7 days and 15 days.

FIG. 10 shows photographs of a cross-section taken at a magnification of 150 times after immersing Example 2 according to an embodiment of the present invention in urine for 1 day, 3 days, 5 days, 7 days and 15 days.

FIG. 11 shows photographs of a cross-section taken at a magnification of 150 times after immersing Example 3 according to an embodiment of the present invention in urine for 1 day, 3 days, 5 days, 7 days and 15 days.

FIG. 12 shows photographs of a cross-section taken at a magnification of 150 times after immersing Example 4 according to an embodiment of the present invention in urine for 1 day, 3 days, 5 days, 7 days and 15 days.

FIG. 13 shows photographs of a cross-section taken at a magnification of 150 times after immersing Comparative Example 1 in urine for 1 day, 3 days, 5 days, 7 days and 15 days.

FIG. 14 shows photographs of a cross-section taken at a magnification of 150 times after immersing Comparative Example 2 in urine for 1 day, 3 days, 5 days, 7 days and 15 days.

### BEST MODE FOR INVENTION

One embodiment of the present invention provides a polymer composite for ureteral insertion 10, the polymer composite including: a polymer substrate 11; and a coating layer 13 provided on at least one surface of the polymer substrate, wherein the coating layer 13 has a C atom in an amount of 78 at. % or greater and 82 at. % or less and an O atom in an amount of 13 at. % or greater and 17 at. % or less, which are detected by an analysis using an XPS analysis method.

According to one embodiment of the present invention, the coating layer 13 may have an N atom in an amount of 0.5 at. % or greater and 2.5 at. % or less and a Si atom in an amount of 2.5 at. % or greater and 4.5 at. % or less, which are detected by an analysis using an XPS analysis method.

According to one embodiment of the present invention, the coating layer 13 may have a maximum peak of C 1s in a range of 280 eV or greater and 290 eV or less, a maximum peak of O 1s in a range of 530 eV or greater and 540 eV or less, a maximum peak of N 1s in a range of 390 eV or greater and 410 eV or less and a maximum peak of Si 2p in a range of 100 eV or greater and 100 eV or less, which are detected by an analysis using an XPS analysis method.

According to one embodiment of the present invention, the coating layer 13 may have a full width at half maximum of the C 1s peak in a range of 1.2 eV or greater and 1.5 eV or less, a full width at half maximum of the O 1s peak in a range of 1.45 eV or greater and 1.6 eV or less and a full width at half maximum of the N 1s peak in a range of 1.5 eV or greater and 1.8 eV or less, which are detected by an analysis using an XPS analysis method.

According to one embodiment of the present invention, the coating layer 13 may have a full width at half maximum of the Si 2p peak detected by an analysis using an XPS analysis method in a range of 1.2 eV or greater and 1.4 eV or less.

According to one embodiment of the present invention, the coating layer 13 may have an average thickness of 0.1 μm or greater and 0.2 μm or less.

According to one embodiment of the present invention, a material of the polymer substrate 11 may be a polyurethane-based resin.

According to one embodiment of the present invention, there is provided a ureteral stent 100 formed with the polymer composite for ureteral insertion 10, wherein the polymer substrate 11 has a tube shape.

According to one embodiment of the present invention, the coating layer 13 may be provided inside the tube.

According to one embodiment of the present invention, the ureteral stent 100 may have an outer diameter of 1.6 mm or greater and 4.0 mm or less.

According to one embodiment of the present invention, the ureteral stent 100 may have an inner diameter of 0.2 mm or greater and 1.7 mm or less.

According to one embodiment of the present invention, the coating layer 13 may have 1 or more and 5 or less layers.

One embodiment of the present invention provides a method for preparing the polymer composite for ureteral insertion 10, the method including: providing a polymer substrate 11 (S10); and providing a coating layer 13 on at least one surface of the polymer substrate 11 by performing plasma deposition for 3 minutes or longer and 5 minutes or shorter under a mixed gas atmosphere including C₂H₂ (S30).

According to one embodiment of the present invention, the mixed gas may further include one selected from the group consisting of an inert gas, O₂ and combinations thereof.

According to one embodiment of the present invention, the inert gas may be one selected from the group consisting of He, Ne, Ar, Kr, Xe, Rn and combinations thereof.

According to one embodiment of the present invention, a voltage applied for plasma generation in the plasma deposition may be 1,000 V or greater and 5,000 V or less.

According to one embodiment of the present invention, a current applied for plasma generation in the plasma deposition may be 0.1 A or greater and 10.0 A or less.

According to one embodiment of the present invention, total energy of plasma generated in the plasma deposition may be 200 J or greater and 30,000 J or less.

According to one embodiment of the present invention, a pressure of the mixed gas atmosphere in the plasma deposition may be 0.1 torr or greater and 10.0 torr or less at 20° C.

According to one embodiment of the present invention, a flow rate of the mixed gas in the plasma deposition may be sccm or greater and 100 sccm or less.

According to one embodiment of the present invention, average molar energy applied to the mixed gas in the plasma deposition may be 1.0×10⁸ J/mol or greater and 1.0×10¹¹ J/mol or less.

### MODE FOR INVENTION

Throughout the specification, a description of a certain part “including” certain components means that it may further include other components, and does not exclude other components unless particularly stated on the contrary.

Throughout the specification, a description of a certain member being placed “on” another member includes not only a case of the certain member being in contact with the another member but a case of still another member being present between the two members.

Throughout the specification, “A and/or B” means “A and B, or A or B”.

Hereinafter, the present invention will be described in more detail.

One embodiment of the present invention provides a polymer composite for ureteral insertion 10, the polymer composite including: a polymer substrate 11; and a coating layer 13 provided on at least one surface of the polymer substrate, wherein the coating layer 13 has a C atom in an amount of 78 at. % or greater and 82 at. % or less and an O atom in an amount of 13 at. % or greater and 17 at. % or less, which are detected by an analysis using an XPS analysis method.

The polymer composite for ureteral insertion 100 according to one embodiment of the present invention may prevent stone substances, which are foreign substances, from being deposited on the surface by improving hydrophobicity of the surface of the polymer substrate 11.

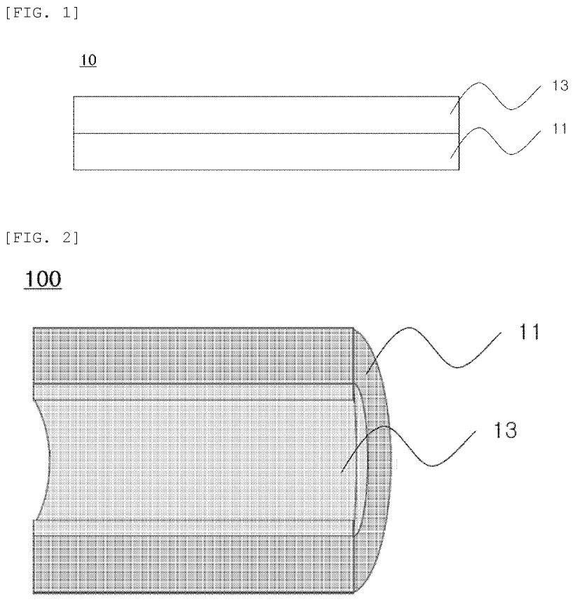

FIG. 1 is a schematic diagram of the polymer composite for ureteral insertion 10 according to one embodiment of the present invention. Referring to FIG. 1, the polymer composite for ureteral insertion 10, which is one embodiment of the present invention, will be specifically described.

According to one embodiment of the present invention, the polymer composite for ureteral insertion 10 includes a polymer substrate 11. By the polymer composite for ureteral insertion 10 including a polymer substrate 11 as described above, the basic shape may be maintained, a coating layer to be described later may be uniformly deposited on the polymer substrate surface to reduce surface roughness, and flexibility of a ureteral stent to be described later may be provided.

According to one embodiment of the present invention, the polymer composite for ureteral insertion 10 includes a coating layer 13. Specifically, the coating layer may be deposited on the surface of the polymer substrate due to plasma generation under a mixed gas atmosphere through plasma deposition as to be described later. By the polymer composite for ureteral insertion 10 including a coating layer 13 as described above, generation of stones and the like may be reduced by increasing hydrophobicity of the surface of a ureteral stent to be described later.

According to one embodiment of the present invention, the polymer composite includes a coating layer 13 provided on at least one surface of the polymer substrate.

Specifically, the coating layer may be provided on one or both surfaces of the polymer substrate, and more specifically, the coating layer may be deposited on one or both surfaces of the polymer substrate. By the polymer composite including a coating layer 13 provided on at least one surface of the polymer substrate as described above, surface roughness of the polymer composite may be reduced, and generation of stones and the like may be reduced by increasing hydrophobic properties of the polymer composite surface.

According to one embodiment of the present invention, the coating layer 13 may have a C atom, which is detected by an analysis using an XPS (X-ray photoelectron spectroscopy) analysis method, in an amount of 78 at. % or greater and 82 at. % or less, 78.5 at. % or greater and 81.5 at. % or less, 78.7 at. % or greater and 81.3 at. % or less or 79 at. % or greater and 80 at. % or less, with respect to the total detected atoms (100 at. %). By adjusting the content of the C atom detected by an analysis using an XPS analysis method in the above-described range in the coating layer 13, cracks occurring in the coating layer may be prevented by controlling stress according to carbon bonding, and generation of stones and the like may be reduced by removing the starting point where foreign substances may be attached by the crack occurrences and improving hydrophobicity of the surface.

According to one embodiment of the present invention, the coating layer 13 may have an O atom, which is detected by an analysis using an XPS analysis method, in an amount of 12.5 at. % or greater and 17.2 at. % or less, 13 at. % or greater and 17 at. % or less, 14 at. % or greater and 17 at. % or less, at. % or greater and 17 at. % or less or 15 at. % or greater and 16.5 at. % or less, with respect to the total detected atoms (100 at. %). By adjusting the content of the O atom detected by an analysis using an XPS analysis method in the above-described range in the coating layer 13, cracks occurring in the coating layer may be prevented by controlling stress occurring in the coating layer, and generation of stones and the like may be reduced by removing the starting point where foreign substances may be attached by the crack occurrences and improving hydrophobicity of the surface.

According to one embodiment of the present invention, the coating layer 13 may have an N atom, which is detected by an analysis using an XPS analysis method, in an amount of 0.5 at. % or greater and 2.5 at. % or less, 0.6 at. % or greater and 2.2 at. % or less, 0.7 at. % or greater and 2.0 at. % or less, 0.8 at. % or greater and 1.7 at. % or less or 0.9 at. or greater and 1.5 at. % or less, with respect to the total detected atoms (100 at. %). By adjusting the content of the N atom detected by an analysis using an XPS analysis method in the above-described range in the coating layer 13, cracks occurring in the coating layer may be prevented by controlling stress occurring in the coating layer, and generation of stones and the like may be reduced by removing the starting point where foreign substances may be attached by the crack occurrences and improving hydrophobicity of the surface.

According to one embodiment of the present invention, the coating layer 13 may have a Si atom, which is detected by an analysis using an XPS analysis method, in an amount of 2.6 at. % or greater and 4.5 at. % or less, 2.7 at. % or greater and 4.0 at. % or less, 2.8 at. % or greater and 3.7 at. % or less or 2.8 at. % or greater and 3.5 at. % or less, with respect to the total detected atoms (100 at. %). By adjusting the content of the Si atom detected by an analysis using an XPS analysis method in the above-described range in the coating layer 13, cracks occurring in the coating layer may be prevented by controlling stress occurring in the coating layer, and generation of stones and the like may be reduced by removing the starting point where foreign substances may be attached by the crack occurrences and improving hydrophobicity of the surface.

According to one embodiment of the present invention, the coating layer 13 may have a maximum peak of C 1s, which is detected by an analysis using an XPS analysis method, in a range of 280 eV or greater and 290 eV or less. By the coating layer 13 exhibiting a maximum peak of C 1s detected by an analysis using an XPS analysis method in the above-described range, bonding relationship with the compounds included in the coating layer may be controlled and generation of stress in the coating layer may be minimized.

According to one embodiment of the present invention, the coating layer 13 may have a maximum peak of O 1s, which is detected by an analysis using an XPS analysis method, in a range of 530 eV or greater and 540 eV or less. By the coating layer 13 exhibiting a maximum peak of O 1s detected by an analysis using an XPS analysis method in the above-described range, bonding relationship with the compounds included in the coating layer may be controlled and generation of stress in the coating layer may be minimized.

According to one embodiment of the present invention, the coating layer 13 may have a maximum peak of N 1s, which is detected by an analysis using an XPS analysis method, in a range of 390 eV or greater and 410 eV or less. By the coating layer 13 exhibiting a maximum peak of N 1s detected by an analysis using an XPS analysis method in the above-described range, bonding relationship with the compounds included in the coating layer may be controlled and generation of stress in the coating layer may be minimized.

According to one embodiment of the present invention, the coating layer 13 may have a maximum peak of Si 2p, which is detected by an analysis using an XPS analysis method, in a range of 100 eV or greater and 100 eV or less. By the coating layer 13 exhibiting a maximum peak of Si 2p detected by an analysis using an XPS analysis method in the above-described range, bonding relationship with the compounds included in the coating layer may be controlled and generation of stress in the coating layer may be minimized.

According to one embodiment of the present invention, the coating layer 13 may have a full width at half maximum (FWHM) of the C 1s peak, which is detected by an analysis using an XPS analysis method, in a range of 1.2 eV or greater and 1.5 eV or less. By the coating layer 13 exhibiting a full width at half maximum of the C 1s peak detected by an analysis using an XPS analysis method in the above-described range, bonding relationship with the compounds included in the coating layer may be controlled and generation of stress in the coating layer may be minimized.

According to one embodiment of the present invention, the coating layer 13 may have a full width at half maximum of the O 1s peak in a range of 1.45 eV or greater and 1.6 eV or less and a full width at half maximum of the N 1s peak in a range of 1.5 eV or greater and 1.8 eV or less, which are detected by an analysis using an XPS analysis method. By the coating layer 13 exhibiting a full width at half maximum of the O 1s peak detected by an analysis using an XPS analysis method in the above-described range, bonding relationship with the compounds included in the coating layer may be controlled and generation of stress in the coating layer may be minimized.

According to one embodiment of the present invention, the coating layer 13 may have a full width at half maximum (FWHM) of the Si 2p peak detected by an analysis using an XPS analysis method in a range of 1.2 eV or greater and 1.4 eV or less. By the coating layer 13 exhibiting a full width at half maximum of the Si 2p peak detected by an analysis using an XPS analysis method in the above-described range, bonding relationship with the compounds included in the coating layer may be controlled and generation of stress in the coating layer may be minimized.

According to one embodiment of the present invention, the coating layer 13 may have an average thickness of 0.1 μm or greater and 2.0 μm or less. By adjusting the average thickness of the coating layer 13 in the above-described range, surface roughness of the coating layer 13 may be controlled, and occurrences of cracks (splits) on the surface caused by an increase in the stress of carbon bonds or peeling-off of the coating layer may be prevented.

According to one embodiment of the present invention, a material of the polymer substrate 11 may be a polyurethane-based resin. Specifically, a material of the polymer substrate 11 may be a polyurethane resin. By selecting a polyurethane-based resin as the polymer substrate 11 as described above, strength and durability of the polymer composite for ureteral insertion may be secured, and the coating layer may be prevented from being peeled off by improving bonding strength with the coating layer.

According to one embodiment of the present invention, there is provided a ureteral stent 100 formed with the polymer composite for ureteral insertion 10, wherein the polymer substrate 11 has a tube shape.

The ureteral stent 100 according to one embodiment of the present invention is capable of improving durability by preventing stone formation due to the coating layer 13 on the inner surface, and may be used for a long time without replacement.

FIG. 2 is a schematic diagram of the ureteral stent 100 according to one embodiment of invention. Referring to FIG. 2, the ureteral stent 100, which is one embodiment of the present invention, will be specifically described.

According to one embodiment of the present invention, the ureteral stent 100 is formed with the polymer composite for ureteral insertion 10. In the present specification, parts overlapping with the description provided for the polymer composite for ureteral insertion 10 will not be included. As the ureteral stent 100 is formed with the polymer composite for ureteral insertion 10 as described above, generation of foreign substances such as stones are minimized on the stent surface even when the stent is inserted into the body, and as a result, blockage of the stent and resulting inflammation, infection and the like may be prevented, and the lifetime of the stent may be extended, minimizing replacement of the stent.

According to one embodiment of the present invention, the polymer substrate 11 of the ureteral stent 100 has a tube shape. Specifically, the tube is shaped like a thin and long hollow tube. Furthermore, a transverse section, which is a diameter direction, of the tube shape may mean a hollow formed in a circular, oval or somewhat distorted shapes thereof, a triangle, a quadrangle and a polygon. By forming the polymer substrate 11 of the ureteral stent 100 in a tube shape as described above, the stent may be inserted into a ureter to enable urine to flow into the tube.

According to one embodiment of the present invention, the coating layer 13 may be provided inside the tube. Specifically, the coating layer may be formed by plasma deposition to be described later inside the polymer substrate having a tube shape. By the coating layer 13 being provided inside the tube as described above, hydrophobicity of the inner surface of the ureteral stent increases, roughness of the inner surface of the ureteral stent is even, and even when the stent is inserted into a ureter and immersed in urine for a long time, deposition of stones and the like, which are foreign substances on the inner surface, may be prevented to improve the lifetime of the stent, and infection and the like caused by the stent may be prevented.

According to one embodiment of the present invention, the ureteral stent 100 may have an outer diameter of 1.6 mm or greater and 4.0 mm or less. The outer diameter may mean, when the ureteral stent 100 is cut in a width direction, the longest distance among distances formed by an outer closed curve and a straight line passing through the inside of the outer closed curve. By adjusting the outer diameter of the ureteral stent 100 in the above-described range, the ureteral stent may be readily inserted into a ureter.

According to one embodiment of the present invention, the ureteral stent 100 may have an inner diameter of 0.2 mm or greater and 1.9 mm or less, and specifically 0.2 mm or greater and 1.7 mm or less. The inner diameter may mean, when the ureteral stent 100 is cut in a width direction, the longest distance among distances formed by an inner closed curve and a straight line passing through the inside of the inner closed curve. By adjusting the inner diameter of the ureteral stent 100 in the above-described range, urine may flow without causing any trouble to the human body when inserting the stent into a ureter.

According to one embodiment of the present invention, the coating layer 13 may have 1 or more and 5 or less layers. Specifically, the coating layer may have a multilayer structure by performing plasma deposition to be described later multiple times. By the coating layer 13 having a multilayer structure of 1 or more and 5 or less layers as described above, flexibility of the ureteral stent 100 may be controlled by adjusting the thickness of the coating layer, and occurrences of cracks and peeling-off of the coating layer may be prevented while controlling internal roughness of the ureteral stent 100.

One embodiment of the present invention provides a method for preparing the polymer composite for ureteral insertion 10, the method including: providing a polymer substrate 11 (S10); and providing a coating layer 13 on at least one surface of the polymer substrate 11 by performing plasma deposition for 3 minutes or longer and 5 minutes or shorter under a mixed gas atmosphere including C₂H₂. Furthermore, when using the polymer substrate having the above-described tube shape, the method for preparing the polymer composite for ureteral insertion 10 may mean a method for manufacturing the ureteral stent.

The method for preparing the polymer composite for ureteral insertion 10 according to one embodiment of the present invention may readily form a coating layer, and, by adjusting the thickness of the coating layer, may prevent stone substances, which are foreign substances, from being deposited on the surface.

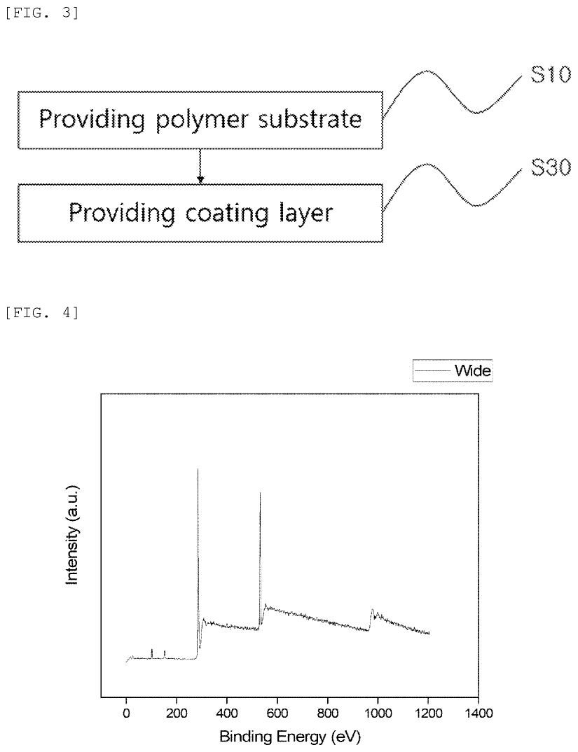

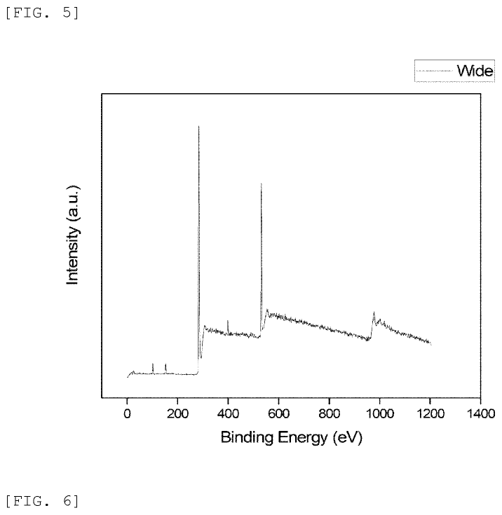

FIG. 3 is a flow chart of the method for preparing the polymer composite for ureteral insertion 10 according to one embodiment of the present invention. Referring to FIG. 3, the method for preparing the polymer composite for ureteral insertion 10 according to one embodiment of the present invention will be specifically described.

According to one embodiment of the present invention, the method includes a step of providing a polymer substrate 11 (S10). Specifically, the polymer substrate may be prepared so that the surface cut to a transverse section in a tube shape as described above has the above-described shape to manufacture the ureteral stent. By including the step of providing a polymer substrate 11 (S10) as described above, the desired shape of the stent may be obtained.

According to one embodiment of the present invention, the method includes a step of providing a coating layer 13 on at least one surface of the polymer substrate 11 by performing plasma deposition for 3 minutes or longer and 5 minutes or shorter under a mixed gas atmosphere including C₂H₂ (S30). By including the step of providing a coating layer 13 on at least one surface of the polymer substrate 11 by performing plasma deposition for 3 minutes or longer and 5 minutes or shorter under a mixed gas atmosphere including C₂H₂ (S30) as described above, components of the coating layer may be controlled, and the peak and the full width at half maximum detected by an analysis using an XPS analysis method may be controlled.

According to one embodiment of the present invention, a coating layer 13 is provided on at least one surface of the polymer substrate 11 by plasma deposition. Specifically, one or both surfaces of the polymer substrate 11 may be provided with a coating layer by plasma deposition. By providing a coating layer 13 on at least one surface of the polymer substrate 11 by plasma deposition as described above, surface roughness of the polymer composite may be reduced, and hydrophobic properties of the surface of the polymer composite may be increased to reduce occurrences of stones and the like.

According to one embodiment of the present invention, the plasma deposition is performed under a mixed gas atmosphere including C₂H₂. By the plasma deposition being performed under a mixed gas atmosphere including C₂H₂ as described above, stress of the coating layer may be controlled by controlling the components included in the coating layer, and hydrophobicity of the surface may be improved, preventing the stent from being blocked due to the generation of stones therein and preventing the coating layer from being peeled off.

According to one embodiment of the present invention, the plasma deposition is performed for 3 minutes or longer and 5 minutes or less. By adjusting the time during which the plasma deposition is performed in the above-described range, the components included in the coating layer may be controlled, the thickness of the coating layer may be controlled, flexibility of the stent may be maintained, the lifetime of the stent may be extended, stress of the coating layer may be controlled, and hydrophobicity of the surface may be improved, preventing the stent from being blocked due to the generation of stones therein and preventing the coating layer from being peeled off.

According to one embodiment of the present invention, the mixed gas may further include one selected from among an inert gas, O₂, N₂ and combinations thereof. By selecting the components of the mixed gas from those described above, carbon bonds included in the coating layer may be controlled, and stress of the coating layer may be controlled, preventing cracks that occur in the coating layer.

According to one embodiment of the present invention, the inert gas may be one selected from the group consisting of He, Ne, Ar, Kr, Xe, Rn and combinations thereof. By selecting the inert gas from those described above, carbon bonds included in the coating layer may be controlled, and stress of the coating layer may be controlled, preventing cracks that occur in the coating layer.

According to one embodiment of the present invention, the plasma deposition may be applied without limit as long as it forms plasma and exposes the polymer substrate to the plasma. Specifically, the plasma deposition may use a plasma-enhanced chemical vapor deposition (PECVD) method. Herein, the plasma may be RF plasma formed using RF (radio frequency) power.

According to one embodiment of the present invention, the voltage applied for plasma generation in the plasma deposition may be 1,000 V or greater and 5,000 V or less. By adjusting the voltage applied for plasma generation in the plasma deposition in the above-described range, occurrences of cracks in the coating layer may be prevented.

According to one embodiment of the present invention, the current applied for plasma generation in the plasma deposition may be 0.1 A or greater and 10.0 V or less. By adjusting the current applied for plasma generation in the plasma deposition in the above-described range, occurrences of cracks in the coating layer may be prevented.

According to one embodiment of the present invention, the frequency in the plasma deposition may be 10 kHz or greater and 100 kHz or less. By adjusting the frequency in the plasma deposition in the above-described range, the thickness of the coating layer may be controlled, and surface roughness of the coating layer may be controlled.

According to one embodiment of the present invention, total energy of plasma generated in the plasma deposition may be 200 J or greater and 30,000 J or less. In this range, occurrences of cracks in the coating layer may be prevented.

According to one embodiment of the present invention, a pressure of the mixed gas atmosphere in the plasma deposition may be 0.1 torr or greater and 10.0 torr or less at 20° C. By adjusting the pressure of the mixed gas atmosphere in the plasma deposition in the above-described range, the thickness of the coating layer and the density of the coating layer may be controlled.

According to one embodiment of the present invention, a flow rate of the mixed gas in the plasma deposition may be 5 sccm or greater and 100 sccm or less. By adjusting the flow rate of the mixed gas in the plasma deposition in the above-described range, the components of the coating layer may be controlled, and the thickness of the coating layer may be controlled.

According to one embodiment of the present invention, average molar energy applied to the mixed gas in the plasma deposition may be 1.0×10⁸ J/mol or greater and 1.0×10¹¹ J/mol or less. By adjusting the average molar energy applied to the mixed gas in the plasma deposition in the above-described range, the components of the coating layer may be controlled, the thickness of the coating layer may be controlled, and occurrences of cracks in the coating layer may be prevented.

Hereinafter, the present invention will be described in detail with reference to examples. However, examples according to the present invention may be modified to various different forms, and the scope of the present invention is not construed as being limited to the examples described below. Examples of the present specification are provided in order to more fully describe the present invention to those having average knowledge in the art.

### Reference Example 1

A polyurethane resin was prepared as a polymer substrate, and the polyurethane resin was made to have a tube shape with an inner diameter of 1.2 mm and an outer diameter of 2.0 mm to prepare a ureteral stent.

### Reference Example 2

A polymer substrate made of a polyurethane resin material having a tube shape with an inner diameter of 1.2 mm and an outer diameter of 2.0 mm was prepared.

After that, plasma deposition was performed for 1 minute under an atmosphere of C₂H₂, O₂ and Ar injected into a plasma deposition apparatus, and by applying a voltage of 2,100 V and adjusting an inner pressure of the plasma deposition apparatus to 0.5 torr to form a coating layer, and as a result, a ureteral stent was manufactured.

### Reference Example 3

A ureteral stent was manufactured in the same manner as in Reference Example 2, except that the plasma deposition was performed for 2 minutes.

### Example 1

A ureteral stent was manufactured in the same manner as in Reference Example 2, except that the plasma deposition was performed for 3 minutes.

### Example 2

A ureteral stent was manufactured in the same manner as in Reference Example 2, except that the plasma deposition was performed for 5 minutes.

### Example 3

A ureteral stent was manufactured in the same manner as in Reference Example 2, except that the plasma deposition was performed for 3 minutes, and a voltage of 2, 600 V was applied to the plasma deposition apparatus.

### Example 4

A ureteral stent was manufactured in the same manner as in Reference Example 2, except that the plasma deposition was performed for 5 minutes, and a voltage of 2, 600 V was applied to the plasma deposition apparatus.

### Comparative Example 1

A ureteral stent was manufactured in the same manner as in Reference Example 2, except that the plasma deposition was performed for 7 minutes, and a voltage of 2, 600 V was applied to the plasma deposition apparatus.

### Comparative Example 2

A ureteral stent was manufactured in the same manner as in Reference Example 2, except that the plasma deposition was performed for 10 minutes, and a voltage of 2,600 V was applied to the plasma deposition apparatus.

### Experimental Example 1 (XPS Analysis)

An XPS (X-ray photoelectron spectroscopy, AXIS SUPRA, Kratos, U.K.) analysis was performed for the inner surface of the ureteral stent of each of Reference Examples 1 to 3 and Examples 1 and 2, and the maximum peak detected by the analysis using the XPS analysis method is summarized in Table 1, and the full width at half maximum of the maximum peak detected by the analysis using the XPS analysis method is summarized in Table 2. In addition, the C, O, N and Si elements detected using the XPS analysis method are summarized as the element ratios (at. %) of C, O, N and Si with respect to the total element ratio (100 at. %) in the following Table 3.

TABLE 1

| | Reference | Reference | Reference | Example | Example |
|---|---|---|---|---|---|
| | Example 1 | Example 2 | Example 3 | 1 | 2 |
| C 1s | 284.50 | 284.50 | 284.51 | 284.50 | 284.50 |
| (Unit: eV) | | | | | |
| 0 1s | 531.80 | 532.04 | 532.11 | 532.01 | 532.08 |
| (Unit: eV) | | | | | |
| N 1s | 399.65 | 399.84 | 399.89 | 399.99 | 399.88 |
| (Unit: eV) | | | | | |
| Si 2p | 101.86 | 102.01 | 102.02 | 102.03 | 102.02 |
| (Unit: eV) | | | | | |

TABLE 2

| | Reference | Reference | Reference | Example | Example |
|---|---|---|---|---|---|
| | Example 1 | Example 2 | Example 3 | 1 | 2 |
| C 1s | 1.18 | 1.15 | 1.11 | 1.27 | 1.26 |
| (Unit: eV) | | | | | |
| 0 1s | 1.20 | 1.44 | 1.42 | 1.50 | 1.50 |
| (Unit: eV) | | | | | |
| N 1s | 1.19 | 1.21 | 1.20 | 1.66 | 1.71 |
| (Unit: eV) | | | | | |
| Si 2p | 1.23 | 1.30 | 1.25 | 1.37 | 1.28 |
| (Unit: eV) | | | | | |

TABLE 3

| | Reference | Reference | Reference | Example | Example |
|---|---|---|---|---|---|
| | Example 1 | Example 2 | Example 3 | 1 | 2 |
| C 1s | 77.89 | 78.53 | 76.59 | 79.70 | 79.64 |
| (Unit: 8) | | | | | |
| 0 1s | 18.41 | 17.39 | 17.49 | 16.26 | 16.08 |
| (Unit: %) | | | | | |
| N 1s | 1.15 | 1.67 | 2.60 | 1.22 | 0.93 |
| (Unit: %) | | | | | |
| Si 2p | 2.55 | 2.41 | 3.32 | 2.81 | 3.35 |
| (Unit: %) | | | | | |

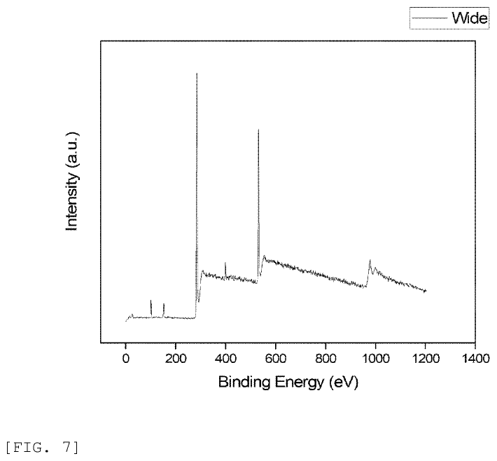

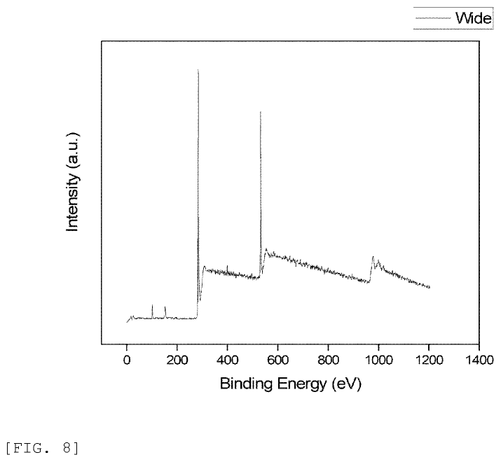

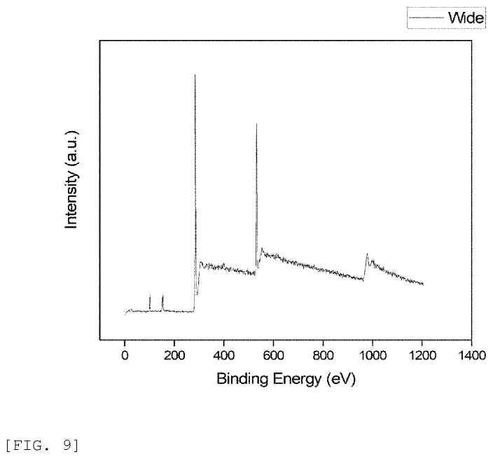

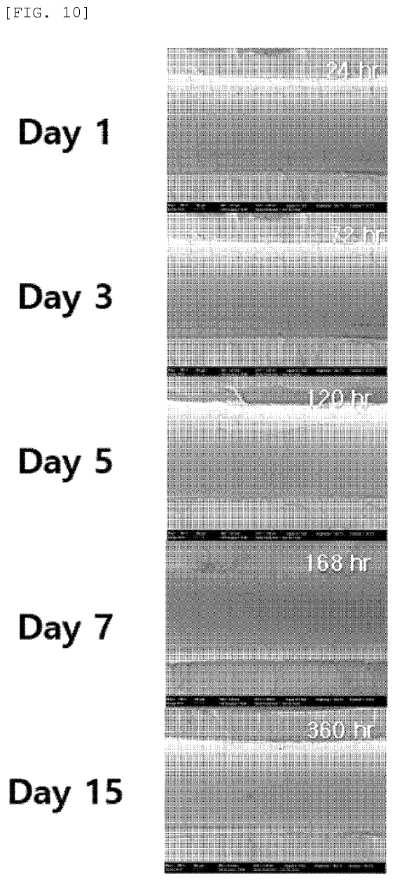

FIG. 4 is a graph showing the XPS analysis result of the polymer substrate layer. FIG. 5 is a graph showing the XPS analysis result of Reference Example 1. FIG. 6 is a graph showing the XPS analysis result of Reference Example 2. FIG. 7 is a graph showing the XPS analysis result of Example 1 according to one embodiment the present invention. FIG. 8 is a graph showing the XPS analysis result of Example 2 according to one embodiment of the present invention.

Referring to FIGS. 4 to 8 and Tables 1 and 2, it was identified that the content of carbon included in the coating layers of Examples 1 and 2 having a long plasma deposition time increased compared to the content of carbon in the polymer substrate that is a polyurethane resin of Reference Example 1 and the content of carbon in Reference Examples 2 and 3 having a short plasma deposition time, and on the contrary, it was identified that the content of oxygen decreased. Through this, it was identified that, as the plasma deposition was performed for 3 minutes or longer and 5 minutes or shorter, bonding configuration was controlled while increasing hydrophobicity of the coating layer, reducing places to which foreign substances may be attached, and occurrences of cracks and peeling-off of the coating layer were able to be prevented by reducing stress of the coating layer.

### Experimental Example 2 (Identification of Crack Occurrences on Coating Layer of Ureteral Stent)

Each of the ureteral stents of Examples 1 to 4 and Comparative Examples 1 and 2 was immersed in human urine for 1 day, 3 days, 5 days, 7 days and 15 days. After that, the ureteral stent was taken out, and cut in a length direction. The inner surface was photographed after magnification to identify occurrences of cracks.

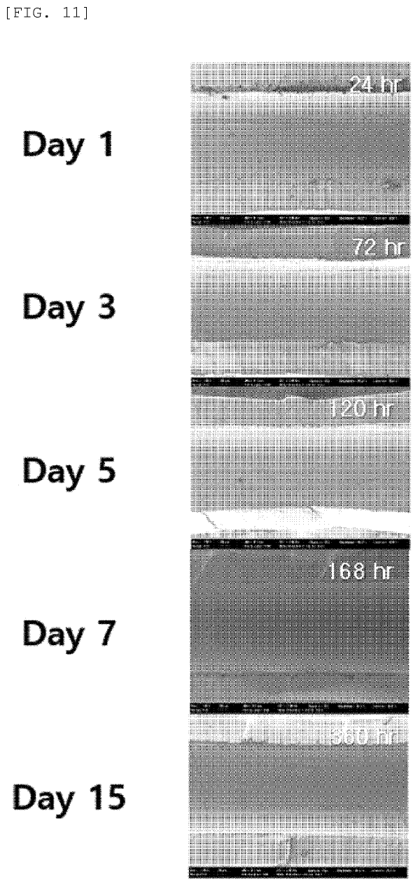

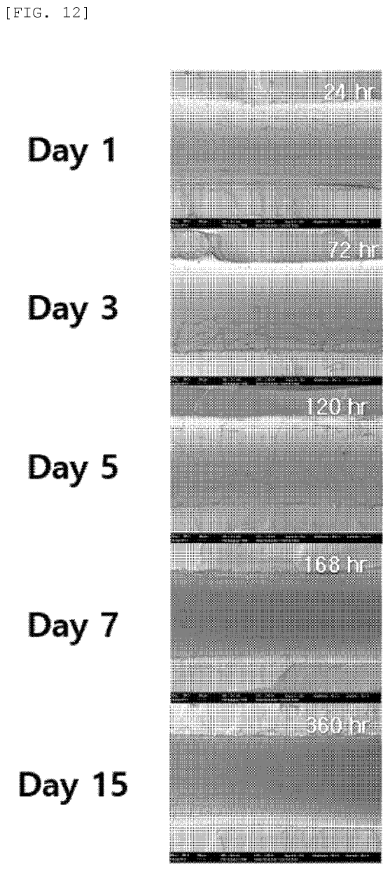

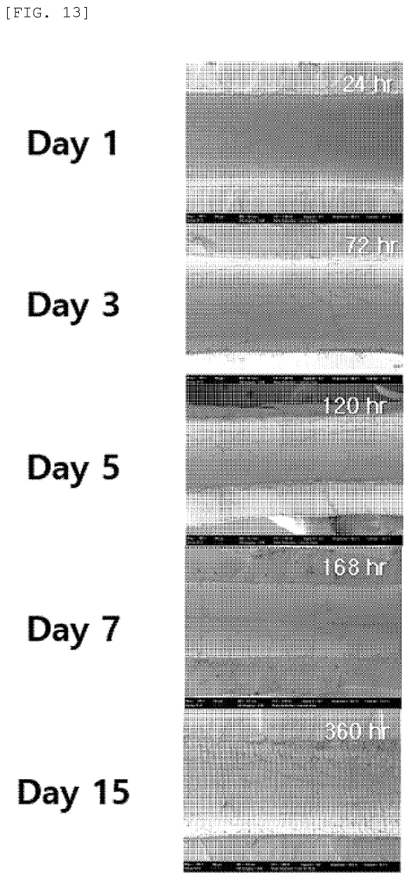

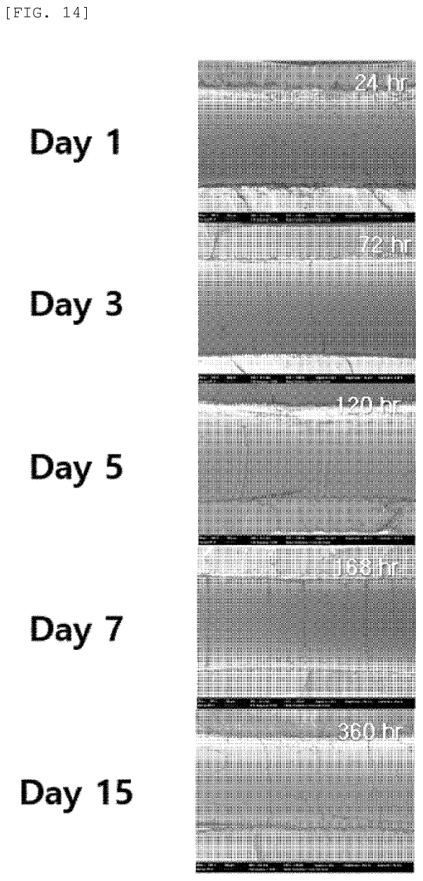

FIG. 9 shows photographs of a cross-section taken at a magnification of 150 times after immersing Example 1 according to an embodiment of the present invention in urine for 1 day, 3 days, 5 days, 7 days and 15 days. FIG. 10 shows photographs of a cross-section taken at a magnification of 150 times after immersing Example 2 according to an embodiment of the present invention in urine for 1 day, 3 days, 5 days, 7 days and 15 days. FIG. 11 shows photographs of a cross-section taken at a magnification of 150 times after immersing Example 3 according to an embodiment of the present invention in urine for 1 day, 3 days, 5 days, 7 days and 15 days. FIG. 12 shows photographs of a cross-section taken at a magnification of 150 times after immersing Example 4 according to an embodiment of the present invention in urine for 1 day, 3 days, 5 days, 7 days and 15 days. FIG. 13 shows photographs of a cross-section taken at a magnification of 150 times after immersing Comparative Example 1 in urine for 1 day, 3 days, 5 days, 7 days and 15 days. FIG. 14 shows photographs of a cross-section taken at a magnification of 150 times after immersing Comparative Example 2 in urine for 1 day, 3 days, 5 days, 7 days and 15 days.

Referring to FIGS. 9 to 13, it was identified that Examples 1 to 4 in which the coating layer was deposited by performing plasma deposition for 3 minutes to 5 minutes had less occurrences of cracks as shown in FIGS. 9 to 12. More specifically, it was identified that Examples 3 and 4 in which the voltage applied for plasma generation was 2600 V and the plasma deposition was performed for 3 minutes to 5 minutes to deposit the coating layer had less occurrences of cracks in the coating layer, and cracks did not increase even when the time for immersion in urine increased. However, it was identified that Comparative Examples 1 and 2 in which the voltage applied for plasma generation was 2600 V and the plasma deposition was performed for longer than 5 minutes had excessive occurrences of cracks in the coating layer, and the cracks increased as the time for immersion in urine increased. Accordingly, it was identified that, due to the excessive occurrences of cracks, foreign substances were attached to the crack sites, resulting in excessive formation of stones.

Furthermore, when comparing Examples 1 and 2 with Examples 3 and 4, it was identified that cracks less occurred in Examples 1 and 2 having a lower applied voltage for plasma generation even when the same plasma deposition was performed.

Accordingly, with the polymer composite for ureteral insertion, the method for preparing the same and the ureteral stent using the same according to one embodiment of the present invention, an appropriate thickness of the coating layer is obtained and occurrences of cracks are minimized when forming the coating layer by adjusting the time for performing the plasma deposition and controlling the condition for the plasma deposition, and as a result, encrustation and biofilm (EBF) formation may be suppressed inside the stent and flexibility of the stent may be maintained.

### Experimental Example 3 (Identification of Attachment of Foreign Substance to Ureteral Stent)

Specifically, each of Reference Example 1, Reference Example 2, Reference Example 3, Example 1, Example 2, Bard Inc. (commercial sample) and Boston Scientific (commercial sample) was immersed in urine of a patient who had undergone a de-identification process, and then stored in an environmental constant temperature chamber at 36.5° C., an average human body temperature. Each of the Reference Examples, Examples and commercial samples was taken out at 6-hour intervals on day 1 and 24-hour intervals from day 1 to day 7, and the sample was taken out lastly on day 15 after starting the experiment. Each of the samples was freeze-dried at −75° C. immediately after being taken out, and attachment of salt ions such as Ca and Mg was identified by SEM (scanning electron microscope) and EDS (energy dispersive spectroscopy). The results are shown in the following Table 4.

Attachment of salt ions was identified from day 1 (6^{th} hour) to day 15 using the method as above, and the case in which attachment of salt ions was identified was expressed as O and the case in which attachment of salt ions was not identified was expressed as X in the following Table 4.

TABLE 4

| | | | | | | Bard, | Boston |
|---|---|---|---|---|---|---|---|
| | | | | | | Inc. | Scientific |
| | Reference | Reference | Reference | | | (Commercial | (Commercial |
| | Example 1 | Example 2 | Example 3 | Example 1 | Example 2 | Sample) | Sample) |
| Day 1 | ◯ | X | X | X | X | X | X |
| (6^{th} | | | | | | | |
| hour) | | | | | | | |
| Day 1 | ◯ | X | X | X | X | ◯ | ◯ |
| (12^{th} | | | | | | | |
| hour) | | | | | | | |
| Day 1 | ◯ | X | X | X | X | ◯ | ◯ |
| (18^{th} | | | | | | | |
| hour) | | | | | | | |
| Day 2 | ◯ | X | X | X | X | ◯ | ◯ |
| Day 3 | ◯ | ◯ | ◯ | X | X | ◯ | ◯ |
| Day 4 | ◯ | ◯ | ◯ | X | X | ◯ | ◯ |
| Day 5 | ◯ | ◯ | ◯ | ◯ | X | ◯ | ◯ |
| Day 7 | ◯ | ◯ | ◯ | ◯ | X | ◯ | ◯ |
| Day 15 | ◯ | ◯ | ◯ | ◯ | X | ◯ | ◯ |

Referring to FIG. 4, it was identified that Reference Example 1 not coated by plasma deposition had salt ions attached thereto from day 1 (6^{th} hour), and Bard Inc. (commercial sample) and Boston Scientific (commercial sample) had salt ions attached thereto from day 1 (12^{th} hour).

Referring to FIG. 4, it was identified that Reference Examples 2 and 3 coated for 1 minute and 2 minutes by plasma deposition had salt ions attached thereto from day 3. In addition, it was identified that Example 1 coated for 3 minutes by plasma deposition had salt ions attached thereto from day 5, and Example 2 coated for 5 minutes by plasma deposition had salt ions not attached thereto until day 15.

Through this, it was identified that, by performing plasma deposition for 3 minutes or longer and 5 minutes or shorter, foreign substances were not attached since bonding configuration of the coating layer was controlled while increasing hydrophobicity of the coating layer.

Hereinbefore, the present invention has been described with limited examples, however, the present invention is not limited thereto, and it is obvious that various changes and modifications may be made by those skilled in the art within technical ideas of the present invention and the range of equivalents of the claims to be described.

### REFERENCE NUMERAL

10: Polymer composite for ureteral insertion
11: Polymer substrate
13: Coating layer
100: Ureteral stent
S10: Providing polymer substrate
S30: Providing coating layer

1. A polymer composite for ureteral insertion, the polymer composite comprising:
a polymer substrate; and
a coating layer provided on at least one surface of the polymer substrate,
wherein the coating layer has a C atom in an amount of 78 at. % or greater and 82 at. % or less and an O atom in an amount of 13 at. % or greater and 17 at. % or less, which are detected by an analysis using an XPS analysis method.

2. The polymer composite of claim 1, wherein the coating layer has an N atom in an amount of 0.5 at. % or greater and 2.5 at. % or less and a Si atom in an amount of 2.5 at. % or greater and 4.5 at. % or less, which are detected by an analysis using an XPS analysis method.

3. The polymer composite of claim 1, wherein the coating layer has a maximum peak of C 1s in a range of 280 eV or greater and 290 eV or less, a maximum peak of O 1s in a range of 530 eV or greater and 540 eV or less, a maximum peak of N 1s in a range of 390 eV or greater and 410 eV or less and a maximum peak of Si 2p in a range of 100 eV or greater and 100 eV or less, which are detected by an analysis using an XPS analysis method.

4. The polymer composite of claim 3, wherein the coating layer has a full width at half maximum of the C 1s peak in a range of 1.2 eV or greater and 1.5 eV or less, a full width at half maximum of the O 1s peak in a range of 1.45 eV or greater and 1.6 eV or less and a full width at half maximum of the N 1s peak in a range of 1.5 eV or greater and 1.8 eV or less, which are detected by an analysis using an XPS analysis method.

5. The polymer composite of claim 4, wherein the coating layer has a full width at half maximum of the Si 2p peak detected by an analysis using an XPS analysis method in a range of 1.2 eV or greater and 1.4 eV or less.

6. The polymer composite of claim 1, wherein the coating layer has an average thickness of 0.1 μm or greater and 2.0 μm or less.

7. The polymer composite of claim 1, wherein a material of the polymer substrate is a polyurethane-based resin.

8. A ureteral stent formed by the polymer composite of claim 1,
wherein the polymer substrate has a tube shape.

9. The ureteral stent of claim 8, wherein the coating layer is provided inside the tube.

10. The ureteral stent of claim 8, which has an outer diameter of 1.6 mm or greater and 4.0 mm or less.

11. The ureteral stent of claim 8, which has an inner diameter of 0.2 mm or greater and 1.9 mm or less.

12. The ureteral stent of claim 8, wherein the coating layer has 1 or more and 5 or less layers.

13. A method for preparing the polymer composite of claim 1, the method comprising:
providing a polymer substrate; and
providing a coating layer on at least one surface of the polymer substrate by performing plasma deposition for 3 minutes or longer and 5 minutes or shorter under a mixed gas atmosphere including C₂H₂.

14. The method of claim 13, wherein the mixed gas further includes one selected from the group consisting of an inert gas, O₂ and combinations thereof.

15. The method of claim 14, wherein the inert gas is one selected from the group consisting of He, Ne, Ar, Kr, Xe, Rn and combinations thereof.

16. The method of claim 13, wherein a voltage applied for plasma generation in the plasma deposition is 1,000 V or greater and 5,000 V or less.

17. The method of claim 13, wherein a current applied for plasma generation in the plasma deposition is 0.1 A or greater and 10.0 A or less.

18. The method of claim 13, wherein total energy of plasma generated in the plasma deposition is 200 J or greater and 30,000 J or less.

19. The method of claim 13, wherein a pressure of the mixed gas atmosphere in the plasma deposition is 0.1 torr or greater and 10.0 torr or less at 20° C.

20. The method of claim 13, wherein a flow rate of the mixed gas in the plasma deposition is 5 sccm or greater and 100 sccm or less.

21. The method of claim 13, wherein average molar energy applied to the mixed gas in the plasma deposition is 1.0×10⁸ J/mol or greater and 1.0×10¹¹ J/mol or less.
